(19) United States
(12) Patent Application Publication
(10) Pub. No.: US 2022/0304357 A1
(43) Pub. Date: Sep. 29, 2022

(54) LACTIC ACID BACTERIA FOR A HEAT-TREATED FOOD PRODUCT FOR STORAGE AT AMBIENT TEMPERATURE

(71) Applicant: Chr. Hansen A/S, Hoersholm (DK)

(72) Inventors: Christian GILLELADEN, Hoersholm (DK); Victoria PREBNER, Hoersholm (DK); Kim Ib SOERENSEN, Hoersholm (DK)

(73) Assignee: Chr. Hansen A/S, Hoersholm (DK)

(21) Appl. No.: 17/642,491

(22) PCT Filed: Sep. 11, 2020
(86) PCT No.: PCT/EP2020/075468

§ 371 Date: Mar. 11, 2022

(30) Foreign Application Priority Data
Sep. 12, 2019 (EP) 19196915.3

(51) Int. Cl. A23L 33/135; A23C 9/127; A23C 3/02; C12N 1/20

(52) U.S. Cl. CPC A23L 33/135; A23C 9/127; A23C 3/02; C12N 1/20; C12N 1/205; A23Y 2240/75; A23Y 2220/29; A23V 2002/00; C12R 2001/225

(57) ABSTRACT

Process for producing an ambient storage food product comprising providing a food product with a pH of between 3.4 and 4.4, subjecting the food product to a heat treatment to obtain a heat treated food product, adding aseptically to the heat treated food product one or more of ambient storage lactic acid bacteria strains to obtain an ambient storage food product, and storing the ambient storage food product at ambient temperature for a period of time, wherein the ambient storage lactic acid bacteria strain is selected from the group consisting of strains: (i) wherein the strain is capable of retaining viability at the end of 20 days at a temperature of 25 #C; and (ii) wherein the pH at most decreases 0.8 units during the storage period; and (iii) wherein the strain is selected from the group consisting of strains of the genus Oenococcus, and mutants thereof.

### FIELD OF THE INVENTION

The present invention relates to lactic acid bacteria, which are suitable for adding to a heat-treated food product with a pH of between 3.4 and 4.4 to be stored at ambient temperature.

### BACKGROUND OF THE INVENTION

In recent years fermented dairy products, such as yogurts, which can be stored, transported, handled and consumed in non-refrigerated conditions, i.e. at ambient temperature, for several months have become widely used. Such yogurts allow the consumer to carry the yogurt with him/her for a period of time without the need for refrigeration in the same manner as is possible for a number of beverages, and hence such yogurts provide a significant convenience advantage for the consumer. In order to obtain such a long-term shelf life at ambient temperature, the yogurt has been heat-treated after completion of the fermentation process to kill or at least inhibit further growth of the bulk of the lactic acid bacteria used in the fermentation process. Live bacteria and further growth of lactic acid bacteria of the starter culture may lead to continued fermentation and result in post-acidification. The heat-treatment may e.g. be a pasteurization process or an Ultra High Temperature (UHT) process. Such yogurts are sometimes referred to as Post Pasteurization Yogurt or as Ambient Yogurt.

Post Pasteurization Yogurt products contain no or only few viable lactic acid bacteria. However, it is desired that Post Pasteurization Yogurt products contain 30 lactic acid bacteria and or probiotic bacteria in order to provide the consumers with the various benefits of such bacteria, e.g. health and food supplement benefits. Of course, the addition of live bacteria to Post Pasteurization Yogurt products to be stored at ambient temperatures introduces the technical problem that the bacteria will propagate to an extent where the yogurt will spoil, e.g. by 35 a decrease of pH due to an increase in concentration of lactic acid resulting from fermentation. In the prior art this technical challenge has been addressed in a number of different ways. For example, the bacteria cultures in the Post Pasteurization Yogurt products have been added to the Post Pasteurization Yogurt products in the form of spores. Also, the bacteria cultures for addition to the Post Pasteurization Yogurt products have been added in the form of powdered, dried, freeze-dried, coated or encapsulated cultures. Furthermore, the bacteria cultures for addition to the Post Pasteurization Yogurt products have been inactivated e.g. by irradiation, microwave treatment, antibiotics, mild pasteurization, chemical agents (inhibitor) or adjustment of pH, water activity or temperature.

WO2009/116864 discloses a dairy product containing spores of probiotic bacteria, wherein the dairy product can be stored at non-refrigerated temperatures for an extended period of time.

WO2004/069156 discloses food products containing probiotic bacteria, which has been inactivated by irradiation, microwave treatment, antibiotics, mild pasteurization and chemical agents (inhibitor).

EP1289380B1 discloses a food product, such as dairy products, containing non-viable Lactobacillus bacteria. The Lactobacillus may be rendered non-viable e.g. by mild heat-treatment, pH adjustment or water activity adjustment.

EP1514553B1 discloses a double-coated lactic acid bacteria powder with high survival rate in the human body, wherein the lactic acid bacteria have been coated doubly by a protein and a polysaccharide.

CN101323850 discloses a process of producing microcapsules of Lactobacillus helveticus in microencapsulated form having a strong heat resistance.

EP0555618B1 discloses a dietary product containing lyophilized lactic acid bacteria.

CN102492643 discloses a Lactobacillus rhamnosus strain GRX19 and its 35 application in a starter culture for producing a fermented milk product containing live Lactobacillus bacteria. The fermented milk product is subjected to heat treatment, e.g. at 70-75° C. for 15-20 seconds, and the Lactobacillus strain is resistant to said heat treatment in that a fraction of the bacteria, e.g. 10exp7 CFU/ml survives the heat treatment. After heat treatment the heat-treated product is filled aseptically into a container and stored at room temperature for e.g. 30 days.

WO2015/169928 discloses a liquid dairy composition suitable for making a foamed dairy product, wherein the composition is shelf-stable under ambient storage conditions, has a pH of between 3.8 and 4.4 and comprises fermented milk, up to 0.12% hydrolyzed whey protein, up to 5% fat, and up to 1% high methylester pectin.

US20100009034 discloses a process of preparing a fermented milk beverage keeping high viable cell count at ambient temperature comprising performing a milk fermentation using a conventional starter culture of lactic acid bacteria, diluting, mixing and sterilizing, and adding Lactobacillus rhamnosus ATCC 53103 to the mixed milk beverage at aseptic conditions.

US20100015285 discloses a process of preparing a direct-acidified milk beverage keeping high viable cell count at ambient temperature comprising performing direct acidification by adjusting the pH to 4.0-4.5 to obtain acidified milk beverage, sterilizing, and adding Lactobacillus rhamnosus ATCC 53103 together with 0.01-0.3% growth promoting factors, e.g. a carbohydrate, to the mixed milk beverage at aseptic conditions.

WO2017/194650 discloses a process for producing an ambient storage food product comprising providing a food product with a pH of between 3.4 and 4.4, subjecting the food product to a heat treatment to obtain a heat treated food product, and adding aseptically to the heat treated food product one or more of ambient storage lactic acid bacteria strains selected from the group consisting of Lactobacillus paracasei, Lactobacillus rhamnosus, Lactobacillus fermenturn and Lactobacillus delbrueckii subsp. bulgaricus, and mutants and variants thereof.

There is a need for further developing improved Post Pasteurization Yogurt products containing viable lactic acid bacteria.

### SUMMARY OF THE INVENTION

The present invention relates to a process for producing an ambient storage food product wherein the food product has been made by fermentation of lactic acid bacteria starter culture, heat treated to inactivate the lactic acid bacteria to prevent or essentially prevent post-acidification, and adding to the heat treated food product strains of the genus Oenococcus and mutants derived thereof.

In one aspect the present invention relates to a process for producing an ambient storage food product comprising providing a food product with a pH of between 3.4 and 4.4, subjecting the food product to a heat treatment so as to reduce the level of bacteria to no more than 1×10exp02 CFU per g to obtain a heat treated food product, adding aseptically to the heat treated food product one or more of ambient storage lactic acid bacteria strains in a total amount of at least 1.0×10exp03 CFU per g to obtain an ambient storage food product, and storing the ambient storage food product at ambient temperature for a period of time,

wherein the ambient storage lactic acid bacteria strain is selected from the group consisting of strains,

(i) wherein the strain when added in an amount of 1.0×10exp07 CFU per g to a fermented milk test product in the form of yogurt obtained by fermentation with a starter culture containing Streptococcus thermophilus and Lactobacillus delbrueckii bulgaricus at a temperature of 43° C. to a pH of 4.3, which has been heat treated at 75° C. for 30 seconds, is capable of retaining viability in an amount of at least 1.0×10exp03 CFU/g at the end of a storage period of the test product of 120 days at a temperature of 25° C., and

(ii) wherein the pH of the test product at most decreases 0.8 units during the storage period, and

(iii) wherein the strain is selected from the group consisting of strains of the genus Oenococcus, and mutants thereof.

The present invention is based on the unexpected experimental finding that bacterial strains of the genus Oenococcus when added to a yogurt product subjected to storage at ambient temperature is capable of retaining viability at a certain level without decreasing the pH level in any significant degree for a period of at least 120 days. This is a surprising finding, since in a milk substrate, lactic acid bacteria will grow on the carbohydrate source available while reducing the pH until the pH reaches a level, where the bacteria is unable to live, so in general lactic acid bacteria in a milk substrate will either be in a growing pH-reducing state or be dead.

In another aspect the present invention relates to an ambient storage food product comprising ambient storage lactic acid bacteria strains, wherein the product has a pH of between 3.4 and 4.4, wherein the product contains at least 1.0×10exp03 CFU per g of the strain, wherein the ambient storage food product is stored at ambient temperature for a period of time, and wherein the lactic acid bacteria strain is selected from the group consisting of strains,

(i) wherein the strain when added in an amount of 1.0×10exp07 CFU per g to a fermented milk test product in the form of yogurt obtained by fermentation with a starter culture containing Streptococcus thermophilus and Lactobacillus 20 delbrueckii bulgaricus at a temperature of 43° C. to a pH of 4.3, which has been heat treated at 75° C. for 30 seconds, is capable of retaining viability in an amount of at least 1.0×10exp03 CFU/g at the end of a storage period of the test product of 120 days at a temperature of 25° C., and

(ii) wherein the pH of the test product at most decreases 0.8 units during the storage period, and

(iii) wherein the strain is selected from the group consisting of strains of the genus Oenococcus, and mutants thereof.

### DETAILED DISCLOSURE OF THE INVENTION

### Process for Producing an Ambient Storage Food Product

The present invention relates to a process for producing an ambient storage food product comprising providing a food product with a pH of between 3.4 and 4.4, subjecting the food product to a heat treatment so as to reduce the level of bacteria to no more than 1×10exp02 CFU per g to obtain a heat treated food product, adding aseptically to the heat treated food product one or more of ambient storage lactic acid bacteria strains in a total amount of at least 1.0×10exp03 CFU per g to obtain an ambient storage food product, and storing the ambient storage food product at ambient temperature for a period of time, wherein the ambient storage lactic acid bacteria strain is selected from the group consisting of strains,

(i) wherein the strain when added in an amount of 1.0×10exp07 CFU per g to a fermented milk test product in the form of yogurt obtained by fermentation with a starter culture containing Streptococcus thermophilus and Lactobacillus delbrueckii bulgaricus at a temperature of 43° C. to a pH of 4.3, which has been heat treated at 75° C. for 30 seconds, is capable of retaining viability in an amount of at least 1.0×10exp03 CFU/g at the end of a storage period of the test product of 120 days at a temperature of 25° C., and

(ii) wherein the pH of the test product at most decreases 0.8 units during the storage period, and

(iii) wherein the strain is selected from the group consisting of strains of the genus Oenococcus, and mutants thereof.

In a preferred embodiment, the ambient storage strain is capable of retaining viability in an amount of at least 1.0×10exp03 CFU/g, preferably at least 5.0×10exp03 CFU/g, more preferably at least 1.0×10exp04 CFU/g, more preferably at least 5.0×10exp04 CFU/g and most preferably at least 1.0×10exp05 CFU/g at the end of the storage period.

Preferably, the pH at most decreases 0.7, preferably 0.6, preferably 0.5, preferably 0.4, preferably 0.3, and most preferably 0.2 during the storage period.

In a preferred embodiment of the invention the strain when added in an amount of 1.0×10exp07 CFU per g to the test product increases to an amount of at least 5.0×10exp07 CFU per g, preferably 7.5×10exp07 CFU per g, and most preferably 1.0×10exp08 CFU per g.

Preferably, the increase of the amount of cells occurs within 45 days of the addition of the strain to the test product, preferably within 40 days, preferably within 35 days, preferably within 30 days, preferably within 25 days, preferably within 20 days, and most preferably within 15 days. Preferably, the amount of cells reaches a maximum within 45 days of the addition of the strain to the test product, preferably within 40 days, preferably within 35 days, preferably within 30 days, preferably within 25 days, preferably within 20 days, and most preferably within 15 days.

In a particular embodiment of the invention, the ambient storage food product is stored at ambient temperature for a period of at least one day, preferably at least 2 days, more preferably at least 3 days, more preferably at least 4 days, more preferably at least 5 days, more preferably at least 6 days, more preferably at least 7 days, more preferably at least 8 days, more preferably at least 9 days, and most preferably at least 10 days.

In particular embodiment of the invention, the Oenococcus strain is selected from the group consisting of an Oenococcus oeni strain, an Oenococcus kitaharae strain, an Oenococcus sicerae strain and mutants thereof. In a particular embodiment of the invention, the Oenococcus strain is selected from the group consisting of an Oenococcus oeni strain and mutants thereof.

In a particular embodiment of the invention, the Oenococcus strain of the invention is selected from the group consisting of the Oenococcus oeni strain deposited as DSM 33144, the Oenococcus oeni strain deposited as DSM 33145, the Oenococcus oeni strain deposited as DSM 33146, the Oenococcus oeni strain deposited as DSM 33147, the Oenococcus oeni strain deposited as DSM 14498, the Oenococcus oeni strain deposited as DSM 15568, the Oenococcus oeni strain deposited as DSM 15569, the Oenococcus oeni strain deposited as DSM 15570, and the Oenococcus oeni strain deposited as DSM 15571, and mutants thereof.

In a particular embodiment of the invention, the Oenococcus strain of the invention is selected from the group consisting of the Oenococcus oeni strain deposited as DSM 33144, the Oenococcus oeni strain deposited as DSM 33145, the Oenococcus oeni strain deposited as DSM 33146, the Oenococcus oeni strain deposited as DSM 33147, and the Oenococcus oeni strain deposited as DSM 14498, and mutants thereof.

In a particular embodiment of the invention, the Oenococcus strain of the invention is a citrate-negative strain. The terms “citrate-negative” is used in the context of the present invention to characterize a strain which when placed in a medium containing a predetermined amount of citric acid is only capable of degrading at most 80% of said citric acid. In particular, the Oenococcus strain of the invention when placed in a medium containing a predetermined amount of citric acid is only capable of degrading at most 70%, preferably at most 60%, more preferably at most 50%, more preferably at most 40%, more preferably at most 30%, more preferably at most 20%, more preferably at most 15%, and most preferably at most 10% of said citric acid.

Citrate-negative Oenococcus strains are described in WO2004/113488, which is included herein by this reference. In a particular embodiment of the invention, the citrate-negative Oenococcus strain of the invention is selected from the group consisting of the Oenococcus oeni strain deposited as DSM15568, the Oenococcus oeni strain deposited as DSM15569, the Oenococcus oeni strain deposited as DSM15570, and the Oenococcus oeni strain deposited as DSM15571 and mutants thereof.

In a particular embodiment of the invention, the Oenococcus strain of the invention is sucrose-positive.

In a particular embodiment of the invention, the Oenococcus strain of the invention is glucose-positive.

In a particular embodiment of the invention, the Oenococcus strain of the invention is galactose-positive.

In a particular embodiment of the invention, the Oenococcus strain of the invention is fructose-positive.

In a particular embodiment of the invention, the Oenococcus strain of the 35 invention is lactose-deficient.

The ability of the Oenococcus strains of the invention to grow on various carbohydrate sources may be tested using the method as described in Example 2.

In a particular embodiment of the process of the invention, the food product with a pH of between 3.4 and 4.4 is a starter culture fermented milk product provided by fermentation of a milk substrate using a starter culture of lactic acid bacteria to obtain the starter culture fermented milk product.

In a particular embodiment of the process of the invention, the starter culture fermented milk product has a protein content of more than 5.1% by weight (w/w).

In a particular embodiment of the process of the invention, the starter culture fermented milk product is not subjected to diluting.

In the following the process of the invention is described in more detail in relation to the process for producing an ambient storage fermented milk product.

The starter culture may be any conventional starter culture of lactic acid bacteria, including single strain culture and culture blends, used for producing a specific type of fermented milk product. In a preferred embodiment of the above 25 process of the invention, the fermentation is carried out so as to obtain a pH of between 3.0 and 5.0, preferably between 3.9 and 4.8, more preferably between 4.0 and 4.6 and most preferably between 4.1 and 4.4.

The heat treatment so as to reduce the level of bacteria of the starter culture to 30 no more than 1.0×10exp02 CFU per g fermented milk is preferably carried out by subjecting the starter culture fermented milk product to a temperature of between 50° C. and 90° C., preferably between 60° C. and 85° C., more preferably between 65° C. and 82° C., and most preferably between 70° C. and 80° C. The heat treatment is preferably carried out for a period of between 10 seconds and 180 seconds, preferably between 12 seconds and 120 seconds, more preferably between 14 seconds and 90 seconds, more preferably between 16 seconds and 60 seconds, more preferably between 18 seconds and 50 seconds and most preferably between 20 and 40 seconds. Preferably, the level of bacteria of the starter culture is reduced to no more than 1.0×10exp01 CFU per g fermented milk, more preferably 0 CFU per g.

Lactic Acid Bacteria Strain for Use in an Ambient Storage Food Product

In one aspect the present invention relates to a lactic acid bacteria strain for use in an ambient storage food product, wherein the product has a pH of between 3.4 and 4.4, wherein the product contains at least 1.0×10exp03 CFU per g of the strain, wherein the ambient storage food product is stored at ambient temperature for a period of time, and wherein the lactic acid bacteria strain is selected from the group consisting of strains,

(i) wherein the strain when added in an amount of 1.0×10exp07 CFU per g to a fermented milk test product in the form of yogurt obtained by fermentation with a starter culture containing Streptococcus thermophilus and Lactobacillus delbrueckii bulgaricus at a temperature of 43° C. to a pH of 4.3, which has been heat treated at 75° C. for 30 seconds, is capable of retaining viability in an amount of at least 1.0×10exp03 CFU/g at the end of a storage period of the test product of 120 days at a temperature of 25° C., and

(ii) wherein the pH of the test product at most decreases 0.8 units during the storage period, and

(iii) wherein the strain is selected from the group consisting of strain of the genus Oenococcus and mutants thereof.

In a particular embodiment of the strain of the present invention, the product is a chemically acidified product.

In a particular embodiment of the strain of the present invention, the product is a fermented milk product obtained by fermentation of a milk substrate using a starter culture of lactic acid bacteria, wherein the product contains no more than 1×10exp02 CFU of the starter culture per g and at least 1×10exp03 CFU per g of the ambient storage lactic acid bacteria strain.

In a particular embodiment of the strain of the present invention, the product is a fermented milk product obtained by fermentation of a milk substrate using a starter culture of lactic acid bacteria, wherein the fermented milk product after fermentation has been subjected to a heat treatment so as to reduce the level of bacteria of the starter culture to no more than 1×10exp02 CFU per g, and wherein after the heat treatment the ambient storage strain of claim 1 has been added aseptically to the heat treated product in an amount of at least 1.0×10exp03 CFU per g. Preferably, the ambient storage strain of the invention has been added aseptically to the heat treated product in an amount of at least 1.0×10exp04 CFU per g, more preferably at least 1.0×10exp05 CFU per g, more preferably at least 1.0×10exp06 CFU per g, more preferably at least 1.0×10exp07 CFU per g, and most preferably at least 1.0×10exp08 CFU per g.

The strain of the genus Oenococcus of the present invention is described above in connection with the process of the invention, to which reference is made.

The strain of the invention may be formulated in a composition comprising one or more of the strains according to the invention. Thus, in one embodiment the strain of the invention is formulated in a composition containing a single strain according to the invention. In another embodiment the strain is formulated in a composition containing two or more strains of the invention. The composition may be in the form of freeze-dried or frozen granules.

Ambient Storage Food Product

In one embodiment the invention relates to an ambient storage food product comprising ambient storage lactic acid bacteria strains, wherein the product has a pH of between 3.4 and 4.4, wherein the product contains at least 1.0×10exp03 CFU per g of the strain, wherein the ambient storage food product is stored at ambient temperature for a period of time, and wherein the lactic acid bacteria strain is selected from the group consisting of strains,

(i) wherein the strain when added in an amount of 1.0×10exp07 CFU per g to a fermented milk test product in the form of yogurt obtained by fermentation with a starter culture containing Streptococcus thermophilus and Lactobacillus delbrueckii bulgaricus at a temperature of 43° C. to a pH of 4.3, which has been heat treated at 75° C. for 30 seconds, is capable of retaining viability in an amount of at least 1.0×10exp03 CFU/g at the end of a storage period of the test product of 120 days at a temperature of 25° C., and

(ii) wherein the pH of the test product at most decreases 0.8 units during the storage period, and

(iii) wherein the strain is selected from the group consisting of strains of the genus Oenococcus and mutants thereof.

In one embodiment the invention relates to an ambient storage food product, wherein the product is a chemically acidified product.

In one embodiment the invention relates to an ambient storage food product, wherein the product is a fermented milk product obtained by fermentation of a milk substrate using a starter culture of lactic acid bacteria, wherein the product contains no more than 1×10exp02 CFU of the starter culture per g and at least 1×10exp03 CFU per g of the ambient storage lactic acid bacteria strain.

In one embodiment the invention relates to an ambient storage food product, wherein the product is a fermented milk product obtained by fermentation of a milk substrate using a starter culture of lactic acid bacteria, wherein the fermented milk product after fermentation has been subjected to a heat treatment so as to reduce the level of bacteria of the starter culture to no more than 1×10exp02 CFU per g, and wherein after the heat treatment the ambient storage strain of claim 1 has been added aseptically to the heat treated product in an amount of at least 1.0×10exp03 CFU per g.

In one embodiment the invention relates to an ambient storage food product, wherein the Oenococcus strain is selected from the group consisting of Oenococcus oeni, Oenococcus kitaharae, Oenococcus sicerae and mutants thereof.

In one embodiment the invention relates to an ambient storage food product, wherein the strain is selected from the group consisting of the Oenococcus oeni strain deposited as DSM 33144, the Oenococcus oeni strain deposited as DSM 33145, the Oenococcus oeni strain deposited as DSM 33146, the Oenococcus oeni strain deposited as DSM 33147, the Oenococcus oeni strain deposited as DSM 14498, the Oenococcus oeni strain deposited as DSM 15568, the Oenococcus oeni strain deposited as DSM 15569, the Oenococcus oeni strain deposited as DSM 15570, and the Oenococcus oeni strain deposited as DSM 15571, and mutants thereof.

In a preferred embodiment of the invention, the ambient storage food product is selected from the group consisting of fermented milk products, chemically acidified milk products, fruit beverage, fermented cereal products, chemically acidified cereal products, soy milk products and any mixture thereof. Preferably the ambient storage food product is a fermented milk product wherein the milk is mammalian milk.

The fermented milk product typically contains protein in a level of between 2.0% by weight to 3.5% by weight. The fermented milk product may also be a low protein product with a protein level of between 1.0% by weight and 2.0% by weight. Alternatively, the fermented milk product may be a high protein product with a protein level of above 3.5% by weight, preferably above 5.1% by weight. In a particular embodiment of the fermented milk product of the invention the product is a mixture of a fermented milk product and a cereal product, e.g. an oat product, wherein the cereal product may be a fermented cereal product, e.g. a fermented oat product.

In a particular embodiment of the invention, the ambient storage food product is a fermented cereal product. The fermented cereal product may be prepared by milling the grains of a cereal biological source material to produce a cereal flour, which is then subjected to fermentation. The fermentation of the cereal flour may be carried out using the same lactic acid bacteria (starter culture) as used for fermentation of a milk substrate as described elsewhere in this application.

In a particular embodiment of the invention, the ambient storage food product is a fruit beverage. The fruit beverages may further contain e.g. oat, soy, almond, whey and/or non-fermented milk, e.g. in the form of milk powder. In a particular embodiment, the fruit beverages of the invention do not contain dairy components, such as milk. In another particular embodiment of the fruit beverage of the invention, the fruit beverage further contains a fermented milk product.

In another embodiment of the invention the ambient storage food product of the invention is a chemically acidified product. The acidification may be carried out using any acidifying agent suitable for adding to food products, such as lactic acid, citric acid, fruit juice, fruit pulp and fruit compound. In a particular embodiment, the ambient storage food product is milk acidified with fruit juice.

In a particular embodiment of the invention, the ambient storage food product is a chemically acidified cereal product. The chemically acidified cereal product may be prepared by milling the grains of a cereal biological source material to produce a cereal flour, which is then used to produce an aqueous suspension, and the pH of the said suspension is then adjusted to a desired level. In a particular embodiment, the ambient storage food product is a cereal food product acidified with a fruit beverage.

Any combination of the above-described elements, aspects and embodiments in all possible variations thereof is encompassed by the invention unless otherwise indicated herein or otherwise clearly contradicted by context.

### Definitions

In connection with the present invention the terms and expressions listed below have the following meaning:

The expression “heat treatment” means any treatment using any temperature, for any period of time and by any means or equipment, which inactivates at least a portion of the bacteria of the starter culture. In this connection the term “inactivate” means any stop, reduction or inhibition of growth of the bacteria, e.g. cell lysing.

The expression “ambient storage” means storage at ambient temperature. The expression “ambient temperature” means the temperature of the surroundings, e.g. room temperature. For example, the ambient temperature may be between 5° C. and 40° C., more particularly between 10° C. and 35° C., more particularly between 15° C. and 30° C., and most particularly between 18° C. and 27° C. The ambient temperature may be controlled, i.e. the temperature is the same over the course of one full day (24 hours), or it may be uncontrolled, i.e. it varies over the course of one full day (24 hours).

The expression “viability” means the bacteria is capable of exhibiting growth (forming a colony) on an MRS agar plate incubated at anaerobic conditions at 30° C. for 3 days. The MRS agar has the following composition (g/l):

Peptone: 10.0

Beef extract: 10.0

Yeast extract: 5.0

Dextrose: 20.0

Polysorbate 80: 1.0

Ammonium Citrate: 2.0

Sodium Acetate: 5.0

Magnesium Sulfate: 0.1

Manganese Sulfate: 0.05

Dipotassium Phosphate: 2.0

Agar: 15.0

The expression “ambient storage lactic acid bacteria strain” means a lactic acid bacteria strain, which when added to a fermented milk product is suitable for ambient storage for a period of time.

The expression “starter culture fermented milk product” means a fermented milk product, which contains the starter culture used to ferment the milk.

The expression “heat treated fermented milk product” means a fermented milk product, which has been subjected to heat treatment.

The expression “ambient storage fermented milk product” means a fermented milk product, which is suitable for ambient storage for a period of time.

The expression “lactic acid bacteria” designates a gram-positive, microaerophilic or anaerobic bacterium, which ferments sugars with the production of acids including lactic acid as the predominantly produced acid, acetic acid and propionic acid. The industrially most useful lactic acid bacteria are found within the order “Lactobacillales” which includes Lactococcus spp., Streptococcus spp., 10 Lactobacillus spp., Leuconostoc spp., Pseudoleuconostoc spp., Pediococcus spp., Brevibacterium spp., Enterococcus spp. and Propionibacterium spp. These are frequently used as food cultures alone or in combination with other lactic acid bacteria.

Lactic acid bacteria, including bacteria of the species Lactobacillus sp. and Lactococcus sp., are normally supplied to the dairy industry either as frozen or freeze-dried cultures for bulk starter propagation or as so-called “Direct Vat Set” (DVS) cultures, intended for direct inoculation into a fermentation vessel or vat for the production of a dairy product, such as a fermented milk product or a cheese. Such lactic acid bacterial cultures are in general referred to as “starter cultures” or “starters”.

The term “milk” is to be understood as the lacteal secretion obtained by milking of any mammal, such as cows, sheep, goats, buffaloes or camels. In a preferred embodiment, the milk is cow's milk. The term milk also includes protein/fat solutions made of plant materials, e.g. soy milk.

The term “milk substrate” may be any raw and/or processed milk material that can be subjected to fermentation according to the method of the invention. Thus, useful milk substrates include, but are not limited to, solutions/-suspensions of any milk or milk like products comprising protein, such as whole or low fat milk, skim milk, buttermilk, reconstituted milk powder, condensed milk, dried milk, whey, whey permeate, lactose, mother liquid from crystallization of lactose, whey protein concentrate, or cream. Obviously, the milk substrate may originate from any mammal, e.g. being substantially pure mammalian milk, or reconstituted milk powder.

Prior to fermentation, the milk substrate may be homogenized and pasteurized according to methods known in the art.

“Homogenizing” as used herein means intensive mixing to obtain a soluble suspension or emulsion. If homogenization is performed prior to fermentation, it may be performed so as to break up the milk fat into smaller sizes so that it no longer separates from the milk. This may be accomplished by forcing the milk at high pressure through small orifices.

“Pasteurizing” as used herein means treatment of the milk substrate to reduce or eliminate the presence of live organisms, such as microorganisms. Preferably, pasteurization is attained by maintaining a specified temperature for a specified period of time. The specified temperature is usually attained by heating. The temperature and duration may be selected in order to kill or inactivate certain bacteria, such as harmful bacteria. A rapid cooling step may follow.

“Fermentation” in the methods of the present invention means the conversion of carbohydrates into alcohols or acids through the action of a microorganism. Preferably, fermentation in the methods of the invention comprises conversion of lactose to lactic acid.

Fermentation processes to be used in production of dairy products are well known and the person of skill in the art will know how to select suitable process conditions, such as temperature, oxygen, amount and characteristics of microorganism(s) and process time. Obviously, fermentation conditions are selected so as to support the achievement of the present invention, i.e. to obtain a dairy product in solid (such as a cheese) or liquid form (such as a fermented milk product).

In the present context, the term “mutant” should be understood as a strain derived from a strain of the invention by means of e.g. genetic engineering, radiation and/or chemical treatment, and/or selection, adaptation, screening, 35 etc. It is preferred that the mutant is a functionally equivalent mutant, e.g. a mutant that has substantially the same, or improved, properties with respect to suitability for ambient storage as the mother strain. Such a mutant is a part of the present invention. Especially, the term “mutant” refers to a strain obtained by subjecting a strain of the invention to any conventionally used mutagenization treatment including treatment with a chemical mutagen such as ethane methane sulphonate (EMS) or N-methyl-N′-nitro-N-nitroguanidine (NTG), UV light or to a spontaneously occurring mutant. A mutant may have been subjected to several mutagenization treatments (a single treatment should be understood one mutagenization step followed by a screening/selection step), but it is presently preferred that no more than 20, no more than 10, or no more than 5, treatments are carried out. In a presently preferred mutant, less than 1%, or less than 0.1%, less than 0.01%, less than 0.001% or even less than 0.0001% of the nucleotides in the bacterial genome have been changed (such as by replacement, insertion, deletion or a combination thereof) compared to the mother strain.

Preferably, the “mutant” of the strain of the present invention has the property that when added in an amount of 1.0×10exp07 CFU per g to a fermented milk test product in the form of yogurt obtained by fermentation with a starter culture containing Streptococcus thermophilus and Lactobacillus delbrueckii bulgaricus at a temperature of 43° C. to a pH of 4.3, which has been heat treated at 75° C. for 30 seconds, the strain is capable of retaining viability in an amount of at least 1.0×10exp03 CFU/g at the end of a storage period of the test product of 120 days at a temperature of 25° C., and wherein the pH at most decreases 0.8 units during the storage period.

Preferably, the “mutant” of the strain of the present invention has less than 25, more preferably less than 10, more preferably less than 9, more preferably less than 8, more preferably less than 7, more preferably less than 6, more preferably less than 5, more preferably less than 4, more preferably less than 3, more preferably less than 2 mutations in the amino acid sequence of one or more of the proteins of the strain. In this connection the term “mutation” means a mutation selected from the group consisting of a substitution, a deletion and an insertion.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The expression “fermented milk product” means a food or feed product wherein the preparation of the food or feed product involves fermentation of a milk substrate with a lactic acid bacteria. “Fermented milk product” as used herein includes but is not limited to products such as thermophilic fermented milk products, e.g. yogurt, mesophilic fermented milk products, e.g. sour cream and buttermilk, cheese as well as fermented whey.

The term “thermophile” herein refers to microorganisms that thrive best at temperatures above 43° C. The industrially most useful thermophilic bacteria include Streptococcus spp. and Lactobacillus spp. The term “thermophilic fermentation” herein refers to fermentation at a temperature above about 35° C., such as between about 35° C. to about 45° C. The term “thermophilic fermented milk product” refers to fermented milk products prepared by thermophilic fermentation of a thermophilic starter culture and include such fermented milk products as set-yogurt, stirred-yogurt and drinking yogurt, e.g. Yakult.

The term “mesophile” herein refers to microorganisms that thrive best at moderate temperatures (15° C.-40° C.). The industrially most useful mesophilic bacteria include Lactococcus spp. and Leuconostoc spp. The term “mesophilic fermentation” herein refers to fermentation at a temperature between about 22° C. and about 35° C. The term “mesophilic fermented milk product” refers to fermented milk products prepared by mesophilic fermentation of a mesophilic starter culture and include such fermented milk products as buttermilk, sour milk, cultured milk, smetana, sour cream, Kefir and fresh cheese, such as quark, tvarog and cream cheese.

The term “cheese” is understood to encompass any cheese, including hard, semi-hard and soft cheeses, such as cheeses of the following types: Cottage, Feta, Cheddar, Parmesan, Mozzarella, Emmentaler, Danbo, Gouda, Edam, Feta-type, blue cheeses, brine cheeses, Camembert and Brie. The person skilled in the art knows how to convert the coagulum into cheese, methods can be found in the literature, see e.g. Kosikowski, F. V., and V. V. Mistry, “Cheese and Fermented Milk Foods”, 1997, 3rd Ed. F. V. Kosikowski, L. L. C. Westport, Conn. As used herein, a cheese which has a NaCl concentration below 1.7% (w/w) is referred to as a “low-salt cheese”.

In the present context the term “fruit juice” refers to the liquid naturally contained in fruit prepared by mechanically squeezing or macerating fresh fruits without the presence of heat and solvents. The “fruit juice” may consist of juice from one type of fruit or a mixture of more than one type of fruit.

The term “fruit drink” in the present context refers to a beverage having a fruit juice content of between 0 to 29%.

The term “nectar” in the present context refers to a beverage having a fruit juice content of between 30 to 99% fruit juice.

In the present context the term “puree” refers to fruits prepared by grounding, pressing and/or straining into the consistency of a thick liquid or a soft paste without the presence of heat and solvents. “Puree” is made of 100% fruit as opposed to being made from just the juice of the fruit.

In the present context the term “fruit beverage” refers to a beverage comprising fruit juice, fruit concentrate and/or fruit puree. The term “fruit beverage” covers “fruit juice”, “fruit drink” and “nectar” as defined herein. The “fruit beverage” may be either one containing pulp, or one from which the pulp has been removed by such an operation as centrifugation.

The term “adding aseptically” means without introducing or introducing a minimum of any microorganism other than the ambient storage lactic acid bacteria.

The term “cereal product” means any product obtained from a cereal or grain biological source material, including oat, corn, barley, rye, buckwheat, wheat and rice.

The term “lactose-deficient” is used in the context of the present invention to characterize LAB which either partially or completely lost the ability to use lactose as a source for cell growth or maintaining cell viability. Such LAB are capable of metabolizing one or several carbohydrates selected from sucrose, galactose and/or glucose or another fermentable carbohydrate. Since these carbohydrates are not naturally present in milk in sufficient amounts to support fermentation by lactose deficient mutants, it will be necessary to add these carbohydrates to the milk. Lactose deficient and partially deficient LAB can be characterized as white colonies on a medium containing lactose and X-Gal.

The term “citrate-negative” strains means strains forming white colonies on a Kempler Mckay agar medium whereas citrate positive strains form dark blue colonies on the said medium, wherein the Kempler Mckay medium is defined in the publication “Improved medium for detection of citrate-fermenting Streptococcus lactis subsp. diacetylactis”, G. M. Kempler and L. L. Mckay, Applied and Environmental Microbiology, April 1980, p 926-927, Vol. 39, No. 4. The Kempler Kakay medium has the following composition:

1% (wt/vol) nonfat milk

0.25% milk protein-hydrolysate peptone

0.5% dextrose

1.5% agar

Method of Preparation:

After sterilization of the medium for 12 minutes at 10 lb/int, it was tempered at 45° C. Two solutions, one containing 10% potassium ferricyanide and one containing 1 g of ferric citrate and 1 g of sodium citrate in 40 ml of water, were steamed (100° C.) for 30 min. Ten milliliters of each solution was added to 1 liter of agar medium, and the agar was swirled gently and poured. Plates were dried in the dark for 24 h at 30° C.

The expression “X.Xx10expYY” and “X.XEYY”, both mean X.Xx10^{YY}, and the two said expressions are used interchangeably.

The expression “CFU” means Colony Forming Units.

### SPECIFIC ITEMS OF THE INVENTION

1. An ambient storage lactic acid bacteria strain, wherein the strain when added in an amount of 1.0×10exp07 CFU per g to a fermented milk test product in the form of yogurt obtained by fermentation with a starter culture containing 20 Streptococcus thermophilus and Lactobacillus delbrueckii bulgaricus at a temperature of 43° C. to a pH of 4.3, which has been heat treated at 75° C. for 30 seconds, is capable of retaining viability in an amount of at least 1.0×10exp03 CFU/g at the end of a storage period of the test product of 120 days at a temperature of 25° C., and wherein the pH at most decreases 0.8 units during the storage period, and wherein the strain is selected from the group consisting of strains of the genus Oenococcus and mutants thereof.

2. Strain according to claim 1, wherein the strain is capable of retaining viability in an amount of at least 1.0×10exp03 CFU/g, preferably at least 5.0×10exp03 CFU/g, more preferably at least 1.0×10exp04 CFU/g, more preferably at least 5.0×10exp04 CFU/g and most preferably at least 1.0×10exp05 CFU/g at the end of the storage period.

3. Strain according to claim 1 or 2, wherein the pH at most decreases 0.7, preferably 0.6, preferably 0.5, preferably 0.4, preferably 0.3, and most preferably 0.2 during the storage period.

4. Strain according to any of the preceding claims, wherein the strain when added in an amount of 1.0×10exp07 CFU per g to the test product increase to an amount of at least 5.0×10exp07 CFU per g, preferably 7.5×10exp07 CFU per g, and most preferably 1.0×10exp08 CFU per g.

5. Strain according to claim 4, wherein the increase of the amount of cells occurs within 45 days of the addition of the strain to the test product, preferably within 40 days, preferably within 35 days, preferably within 30 days, preferably within 25 days, preferably within 20 days, and most preferably within 15 days.

6. Strain according to claim 4 or 5, wherein the amount of cells reaches a maximum within 45 days of the addition of the strain to the test product, preferably within 40 days, preferably within 35 days, preferably within 30 days, preferably within 25 days, preferably within 20 days, and most preferably within 15 days.

7. Composition comprising one or more of the ambient storage lactic acid bacteria strains according to any of claim 1-6.

8. Ambient storage food product with a pH of between 3.4 and 4.4, wherein the product contains at least 1.0×10exp03 CFU per g of the ambient storage strain of claim 1.

9. Food product according to claim 8, wherein product is a chemically acidified product.

10. Food product according to claim 8, wherein the product is a fermented milk product obtained by fermentation of a milk substrate using a starter culture of lactic acid bacteria, wherein the product contains no more than 1×10exp02 CFU of the starter culture per g and at least 1×10exp03 CFU per g of the ambient storage strains of claim 1.

11. Food product according to claim 8, wherein the product is a fermented milk product obtained by fermentation of a milk substrate using a starter culture of lactic acid bacteria, wherein the fermented milk product after fermentation has been subjected to a heat treatment so as to reduce the level of bacteria of the starter culture to no more than 1×10exp02 CFU per g, and wherein after the heat treatment the ambient storage strain of claim 1 has been added aseptically to the heat treated product in an amount of at least 1.0×10exp03 CFU per g.

12. Process for producing an ambient storage food product comprising providing a food product with a pH of between 3.4 and 4.4, subjecting the food product to a heat treatment so as to reduce the level of bacteria to no more than 1×10exp02 CFU per g to obtain a heat treated food product, and adding aseptically to the heat treated food product one or more of the ambient storage lactic acid bacteria strains of claim 1 in a total amount of at least 1.0×10exp03 CFU per g to obtain an ambient storage food product.

13. Process for producing an ambient storage fermented milk product comprising fermentation of a milk substrate using a starter culture of lactic acid bacteria to obtain a starter culture fermented milk product, subjecting the starter culture fermented milk product to a heat treatment so as to reduce the level of bacteria of the starter culture to no more than 1×10exp02 CFU per g to obtain a heat treated fermented milk product, and adding aseptically to the heat treated fermented milk product one or more of the ambient storage lactic acid strains of claim 1 in a total amount of at least 1.0×10exp03 CFU per g to obtain an ambient storage fermented milk product.

14. Use of an ambient storage lactic acid bacteria strain according to claim 1 for adding aseptically to a heat treated food product in a total amount of at least 1.0×10exp03 CFU per g, wherein the heat treated food product has a pH of between 3.4 and 4.4 and has been subjected to a heat treatment so as to reduce the level of starter culture bacteria to no more than 1×10exp02 CFU per g.

15. A lactic acid bacteria strain, wherein the strain is selected from the group consisting of the Oenococcus oeni strain deposited as DSM 33144, the Oenococcus oeni strain deposited as DSM 33145, the Oenococcus oeni strain deposited as DSM 33146, the Oenococcus oeni strain deposited as DSM 33147, and the Oenococcus oeni strain deposited as DSM 14498, and mutants thereof.

### EXAMPLES

### Example 1: Testing of Ambient Storage Suitability of Six Oenococcus oeni Strains in Post-Pasteurized Yogurt (PPY)

TABLE 1
Milk substrate

| Ingredient | Specification | Dosage (%) |
|---|---|---|
| Fresh milk | Whole milk 3.5% fat | 74.3 |
| Added carbohydrate | Sucrose | 7.00 |
| Whey Protein | Nutrilac YO-7830, Aria | 0.60 |
| Concentrate WPC 80 | Food Ingredients | |
| Modified starch | Clearam CJ5025, Roquette | 1.5 |
| Pectin | LM-106 AS-YA, CP Kelco | 0.12 |
| Gellan gum | Kelcogel YSS, CP Kelco | 0.03 |
| Water | Water | 16.45 |

Composition of final post-pasteurized yogurt

Fat: 2.6%

Protein: 3.0%

Carbohydrates: 11.4%

Starter culture: Commercial YoFlex starter culture type FD-DVS YF-L904. The starter culture was inoculated into the milk base at a level of 500 U/2500 L milk.

Ambient storage strains: Six commercial Oenococcus oeni strains were tested.

Each strain was inoculated into the post-pasteurized yogurt at a level of

1×10exp07 cfu/g.

Test strain 1: DSM33146

Test strain 2: DSM33147

Test strain 3: DSM15570

Test strain 4: DSM33144

Test strain 5: DSM33145

Test strain 6: DSM14498

Procedure for Producing Test Product

1. Dispersing the dry ingredients into the milk

2. Resting for minimum 2 hours at 10° C. with gentle stirring

3. Heating the milk until a temperature of 65° C. is reached

4. Homogenization at 150 Bar

5. Heat treatment to 95° C. for 5 min.

6. Cooling to fermentation temperature 43° C.

7. Pump the milk into fermentation vat

8. Inoculation of YoFlex Culture FD-DVS YF-L904.

9. Fermentation until pH reaches 4.30.

10. Break the curd and stir until smooth structure is obtained

11. Heat treatment at 75° C. for 30 sec.

12. Post treatment at 2 bar.

13. Cooling to 25° C.

14. Aseptic filling into 100 ml sterile containers.

Procedure for Testing Ambient Storage Strains

15. Inoculation of strains & cultures

16. Storage for 120 days at room temperature 23° C.

Measurement of pH

Calibration of pH electrode with standard buffer solutions pH 4.01; pH 7.00 and pH 9.21, using temperature compensation. Samples are measured at the same temperature, in this case room temperature (23° C.). Displayed measurement must have a stable signal for at least 30 s, then value is recorded. Electrode is rinsed with deionized water and carefully wiped with a soft paper tissue in between the samples.

pH is measured at day +0 and day +1, and then followed by monthly measurements until day +120.

Method of Cell Counting

Cell population is monitored by plate counts of colonies (cfu/g), where cultivation is conducted on De Man, Rogosa and Sharpe (MRS) media, adjusted to pH 5.4. Incubate samples at 30° C. for 10 days under anaerobic conditions. On MRS media, colonies are visible as small, round and light colonies.

Cell count is analyzed at day +0 and day +1, and then followed by monthly measurements until day +120.

MRS Medium

The MRS agar has the following composition (g/l):

Peptone: 10.0

Beef extract: 10.0

Yeast extract: 5.0

Dextrose: 20.0

Polysorbate 80: 1.0

Ammonium Citrate: 2.0

Sodium Acetate: 5.0

Magnesium Sulfate: 0.1

Manganese Sulfate: 0.05

Dipotassium Phosphate: 2.0

Agar: 15.0

Results

TABLE 2
pH

| Test | | | | | | |
|---|---|---|---|---|---|---|
| strain | Day 0 | Day 1 | Day 30 | Day 60 | Day 90 | Day 120 |
| 1 | 4.29 | 4.30 | 4.25 | 4.22 | 4.22 | 4.19 |
| 2 | 4.29 | 4.30 | 4.27 | 4.24 | 4.22 | 4.22 |
| 3 | 4.29 | 4.30 | 4.19 | 4.14 | 4.13 | 4.09 |
| 4 | 4.29 | 4.30 | 4.30 | 4.30 | 4.29 | 4.32 |
| 5 | 4.29 | 4.31 | 4.29 | 4.22 | 4.20 | 4.20 |
| 6 | 4.29 | 4.30 | 4.30 | 4.23 | 4.20 | 4.20 |

TABLE 3
Cell counts

| Test | | | | | | |
|---|---|---|---|---|---|---|
| strain | Day 0 | Day 1 | Day 30 | Day 60 | Day 90 | Day 120 |
| 1 | 6.60E06 | 7.90E06 | 2.00E08 | 1.20E08 | 4.70E07 | 5.30E07 |
| 2 | 7.20E06 | 6.10E06 | 1.60E05 | 4.32E07 | 5.50E07 | 4.70E07 |
| 3 | 7.80E06 | 7.90E06 | <1.00E04 | 2.56E07 | 5.20E07 | 6.50E07 |
| 4 | 4.60E06 | 4.50E06 | <1.00E04 | 1.44E07 | 2.70E07 | 4.60E07 |
| 5 | 1.55E07 | 1.80E07 | 2.00E08 | 2.20E08 | 1.10E08 | 1.01E08 |
| 6 | 7.20E06 | 1.17E07 | 1.35E08 | 1.90E08 | 1.70E08 | 8.90E07 |

As will appear from Table 2, for five out of six test strains the level of post-acidification is below 0.10 pH units for a storage period of 120 days at 25° C., whereas for the sixth test strain the level of post-acidification is below 0.20 pH units. Thus, all six Oenococcus oeni strains tested only give rise to an extremely low level of post-acidification.

As will appear from Table 3, for all six test strains the cell count after 120 days of storage is higher than at the time of inoculation at day 0. It is assumed that the reason for this is that the tested Oenococcus oeni cultures have had a certain level of growth during the storage period. If so, the said growth has at most resulted in a very limited pH decrease.

### Example 2: Testing of Oenococcus oeni Strains for Growth on Different Carbohydrate Sources

A commercial test named “Api 50 CHL Medium” from bioMérieux SA was used to test the ability of the following Oenococcus strains of the invention to grow on galactose, glucose, fructose, and lactose:

Test strain 2: DSM33147

Test strain 3: DSM15570

Test strain 5: DSM33145

Test Procedure Before Transfer to API Test Strip
Strains have been ordered from the culture collection (from a −80 C stock) and streaked onto GJ5 agar plates
The plates have been incubated anaerobically at 30 C for 7 days
After growth, the plates are checked for purity of the colonies
Afterwards, 2 ml of the API CHL medium are pipetted onto the agar plates
With a sterile spatula, the colonies are scraped off the agar and the concentrated bacterial suspension is transferred into more API CHL medium

Test Procedure in API Test Strip
Of these concentrated solutions, ca. 100 ul are pipetted into the wells of the API test
Paraffin oil has been placed over the well to create a barrier and anaerobiosis
The API strips are incubated at 25 C
The results are read by registering the change in color of the wells after 1 and 4 days.

TABLE 4
Growth on various carbohydrates.

| | Test strain 3 | | | | Test strain 2 | | | | Test stain 5 | | | |
|---|---|---|---|---|---|---|---|---|---|---|---|---|
| | Sample | | | | | | | | | | | |
| | 1 | | 2 | | 1 | | 2 | | 1 | | 2 | |
| | Day | | | | | | | | | | | |
| | 1 | 4 | 1 | 4 | 1 | 4 | 1 | 4 | 1 | 4 | 1 | 4 |
| Neg control | − | − | − | − | − | − | − | − | − | − | − | − |
| Galactose | + | + | + | + | + | + | + | + | − | + | − | + |
| Glucose | + | + | + | + | + | + | + | + | + | + | + | + |
| Fructose | + | + | + | + | + | + | + | + | + | + | + | + |
| D-lactose | − | − | − | − | − | + | − | + | − | − | − | − |

### DEPOSIT AND EXPERT SOLUTION

The Applicant requests that a sample of the deposited microorganisms stated below may only be made available to an expert, until the date on which the patent is granted. In particular, the Applicant requests that the availability of the deposited microorganism referred to in Rule 33 EPC shall be effected only by the issue of a sample to an independent expert nominated by the requester (Rule 32(1) EPC).

TABLE 5
Deposits made by the Applicant, CHR. HANSEN A/S, at a Depositary
Institution having acquired the status of International Depositary
Authority under the Budapest Treaty on the International Recognition
of the Deposit of Microorganisms for the Purposes of Patent Procedure:
Leibniz Institute DSMZ-German Collection of Microorganisms and Cell
Cultures Inhoffenstr. 7B, 38124 Braunschweig, Germany.

| Strain | Accession No. | Deposit date |
|---|---|---|
| Oenococcus oeni | DSM 33144 | 2019 May 28 |
| Oenococcus oeni | DSM 33145 | 2019 May 28 |
| Oenococcus oeni | DSM 33146 | 2019 May 28 |
| Oenococcus oeni | DSM 33147 | 2019 May 28 |

1. Process for producing an ambient storage food product comprising:
providing a food product with a pH of between 3.4 and 4.4,
subjecting the food product to a heat treatment so as to reduce the level of bacteria to no more than 1×10exp02 CFU per g to obtain a heat treated food product,
adding aseptically to the heat treated food product one or more of ambient storage lactic acid bacteria strains in a total amount of at least 1.0×10exp03 CFU per g to obtain an ambient storage food product, and
storing the ambient storage food product at ambient temperature for a period of time,
wherein the ambient storage lactic acid bacteria strain is selected from the group consisting of strains,
(i) wherein the strain when added in an amount of 1.0×10exp07 CFU per g to a fermented milk test product in the form of yogurt obtained by fermentation with a starter culture containing Streptococcus thermophilus and Lactobacillus delbrueckii bulgaricus at a temperature of 43° C. to a pH of 4.3, which has been heat treated at 75° C. for 30 seconds, is capable of retaining viability in an amount of at least 1.0×10exp03 CFU/g at the end of a storage period of the test product of 120 days at a temperature of 25° C., and
(ii) wherein the pH of the test product at most decreases 0.8 units during the storage period, and
(iii) wherein the strain is selected from the group consisting of strains of the genus Oenococcus and mutants thereof.

2. Process according to claim 1, wherein the ambient storage lactic acid bacteria strain is capable of retaining viability in an amount of at least 1.0×10exp03 CFU/g, preferably at least 5.0×10exp03 CFU/g, more preferably at least 1.0×10exp04 CFU/g, more preferably at least 5.0×10exp04 CFU/g and most preferably at least 1.0×10exp05 CFU/g at the end of the storage period of the test product of 120 days.

3. Process according to claim 1 or 2, wherein the pH at most decreases 0.7, preferably 0.6, preferably 0.5, preferably 0.4, preferably 0.3, and most preferably 0.2 during the storage period of the test product of 120 days.

4. Process according to any of the preceding claims, wherein the strain when added in an amount of 1.0×10exp07 CFU per g to the test product increases to an amount of at least 5.0×10exp07 CFU per g, preferably 7.5×10exp07 CFU per g, and most preferably 1.0×10exp08 CFU per g.

5. Process according to claim 4, wherein the increase of the amount of cells occurs within 45 days of the addition of the strain to the test product, preferably within 40 days, preferably within 35 days, preferably within 30 days, preferably within 25 days, preferably within 20 days, and most preferably within 15 days.

6. Process according to claim 4 or 5, wherein the amount of cells reaches a maximum within 45 days of the addition of the strain to the test product, preferably within 40 days, preferably within 35 days, preferably within 30 days, preferably within 25 days, preferably within 20 days, and most preferably within 15 days.

7. Process according to any of claims 1-6, wherein the ambient storage food product is stored at ambient temperature for a period of at least one day, preferably at least 2 days, more preferably at least 3 days, more preferably at least 4 days, more preferably at least 5 days, more preferably at least 6 days, more preferably at least 7 days, more preferably at least 8 days, more preferably at least 9 days, and most preferably at least 10 days.

8. Process according to any of claims 1-7, wherein the Oenococcus strain is selected from the group consisting of Oenococcus oeni, Oenococcus kitaharae, Oenococcus sicerae and mutants thereof.

9. Process according to claim 8, wherein the Oenococcus strain is selected from the group consisting of Oenococcus oeni and mutants thereof.

10. Process according to claim 9, wherein the Oenococcus strain is selected from the group consisting of the Oenococcus oeni strain deposited as DSM 33144, the Oenococcus oeni strain deposited as DSM 33145, the Oenococcus oeni strain deposited as DSM 33146, the Oenococcus oeni strain deposited as DSM 33147, the Oenococcus oeni strain deposited as DSM 14498, the Oenococcus oeni strain deposited as DSM 15568, the Oenococcus oeni strain deposited as DSM 15569, the Oenococcus oeni strain deposited as DSM 15570, and the Oenococcus oeni strain deposited as DSM 15571, and mutants thereof.

11. Process according to any of claims 1-10, wherein the food product with a pH of between 3.4 and 4.4 is a starter culture fermented milk product provided by fermentation of a milk substrate using a starter culture of lactic acid bacteria to obtain the starter culture fermented milk product.

12. Process according to any of claims 1-11, wherein the starter culture fermented milk product has a protein content of more than 5.1% by weight.

13. Process according to any of claims 1-12, wherein the starter culture fermented milk product is not subjected to diluting.

14. An ambient storage food product comprising ambient storage lactic acid bacteria strains, wherein the product has a pH of between 3.4 and 4.4, wherein the product contains at least 1.0×10exp03 CFU per g of the strain, wherein the ambient storage food product is stored at ambient temperature for a period of time, and
wherein the ambient storage lactic acid bacteria strain is selected from the group consisting of strains,
(i) wherein the strain when added in an amount of 1.0×10exp07 CFU per g to a fermented milk test product in the form of yogurt obtained by fermentation with a starter culture containing Streptococcus thermophilus and Lactobacillus delbrueckii bulgaricus at a temperature of 43° C. to a pH of 4.3, which has been heat treated at 75° C. for 30 seconds, is capable of retaining viability in an amount of at least 1.0×10exp03 CFU/g at the end of a storage period of the test product of 120 days at a temperature of 25° C., and
(ii) wherein the pH of the test product at most decreases 0.8 units during the storage period, and
(iii) wherein the strain is selected from the group consisting of strains of the genus Oenococcus and mutants thereof.

15. An ambient storage food product according to claim 14, wherein the product is a chemically acidified product.

16. The ambient storage food product according to claim 14, wherein the product is a fermented milk product obtained by fermentation of a milk substrate using a starter culture of lactic acid bacteria, wherein the product contains no more than 1×10exp02 CFU of the starter culture per g and at least 1×10exp03 CFU per g of the ambient storage lactic acid bacteria strain.

17. The ambient storage food product according to claim 14, wherein the product is a fermented milk product obtained by fermentation of a milk substrate using a starter culture of lactic acid bacteria, wherein the fermented milk product after fermentation has been subjected to a heat treatment so as to reduce the level of bacteria of the starter culture to no more than 1×10exp02 CFU per g, and wherein after the heat treatment the ambient storage strain of claim 1 has been added aseptically to the heat treated product in an amount of at least 1.0×10exp03 CFU per g.

18. The ambient storage food product according to any of claims 14-17, wherein the Oenococcus strain is selected from the group consisting of Oenococcus oeni, Oenococcus kitaharae, Oenococcus sicerae and mutants thereof.

19. The ambient storage food product according to any of claims 14-17, wherein the strain is selected from the group consisting of the Oenococcus oeni strain deposited as DSM 33144, the Oenococcus oeni strain deposited as DSM 33145, the Oenococcus oeni strain deposited as DSM 33146, the Oenococcus oeni strain deposited as DSM 33147, the Oenococcus oeni strain deposited as DSM 14498, the Oenococcus oeni strain deposited as DSM 15568, the Oenococcus oeni strain deposited as DSM 15569, the Oenococcus oeni strain deposited as DSM 15570, and the Oenococcus oeni strain deposited as DSM 15571, and mutants thereof.
